(19) United States
(12) Patent Application Publication
(10) Pub. No.: US 2016/0249974 A1
(43) Pub. Date: Sep. 1, 2016

(54) COMBO DEVICE-SEALER-TRANSCOLLATOR-CUTTER-DISECTOR-TISSUE MANIPULATOR

(71) Applicant: MEDTRONIC ADVANCED ENERGY LLC, Minneapolis, MN (US)

(72) Inventors: Tony Asher, Charlotte, NC (US); Eliot F. Bloom, Hopkinton, NH (US)

(73) Assignee: MEDTRONIC ADVANCED ENERGY LLC, Minneapolis, MN (US)

(21) Appl. No.: 14/955,941

(22) Filed: Dec. 1, 2015

Related U.S. Application Data
(60) Provisional application No. 62/126,224, filed on Feb. 27, 2015.

(51) Int. Cl. A61B 18/14

(52) U.S. Cl. CPC A61B 18/1445; A61B 2018/1457; A61B 2018/1462

(57) ABSTRACT

An electrosurgical forceps device, including a first arm having a first distal end. A second arm having a second distal end is included, the first arm and the second arm being pivotally coupled together. The first arm includes a first electrode at the first distal end and the second arm includes a second electrode at the second distal end. The first electrode and the second electrode are configured to deliver bipolar radiofrequency energy to the tissue. At least one moveable finger grip is disposed on at least one the first arm and the second arm. The device is transitionable between a first configuration, in which the first arm and the second arm are configured to grasp tissue between the first end and the second end, and a second configuration, in which at least one of the first arm and the second arm defines scissors configured to mechanically cut tissue.

### CROSS-REFERENCE TO RELATED APPLICATION

This application is related to and claims priority to U.S. Provisional Patent Application Ser. No. 62/126224, filed Feb. 27, 2015 entitled COMBO DEVICE-SEALER-TRANSCOLLATOR-CUTTER-DISECTOR-TISSUE MANIPULATOR, the entirety of which is incorporated herein by reference.

### STATEMENT REGARDING FEDERALLY SPONSORED RESEARCH OR DEVELOPMENT

n/a

### FIELD OF THE INVENTION

The present invention relates to a medical device for grasping, sealing, cutting, manipulation, and coagulating tissue during a medical procedure.

### BACKGROUND OF THE INVENTION

Surgical forceps are known in the art for grasping and holding tissue during a medical procedure. Such forceps typically include a pair of opposable jaws that pivot about a pivot point at the proximal end of the forceps. However, should a surgeon be desirous to mechanically cut tissue during a procedure, a separate cutting device is often used to mechanically remove the target tissue. Thus, the surgeon often must stop a procedure using forceps to use another device or have another user use scissors to excise tissue, thus raising the time, cost, and efficacy of the medical procedure.

### SUMMARY

The present application advantageously provides for an electrosurgical forceps device, including a first arm having a first distal end. A second arm having a second distal end is included, the first arm and the second arm being pivotally coupled together. The first arm includes a first electrode at the first distal end and the second arm includes a second electrode at the second distal end. The first electrode and the second electrode are configured to deliver bipolar radiofrequency energy to the tissue. At least one moveable finger grip is disposed on at least one the first arm and the second arm. The device is transitionable between a first configuration, in which the first arm and the second arm are configured to grasp tissue between the first end and the second end, and a second configuration, in which at least one of the first arm and the second arm defines scissors configured to mechanically cut tissue.

In another configuration, the electrosurgical forceps device includes a first arm having a first distal end and a second arm having a second distal end, the first arm and the second arm are pivotally coupled together. The first arm includes a first electrode at the first distal end and the second arm includes a second electrode at the second distal end, the first electrode and the second electrode are configured to deliver bipolar radiofrequency energy to the tissue. A scissors assembly movably coupled to at least a portion of the first arm and at least a portion of the second arm is included.

In yet another embodiment, the electrosurgical forceps includes a first arm having a first distal end and a second arm having a second distal end, the first arm and the second arm being pivotally coupled together. The first arm includes a first electrode at the first distal end and the second arm including a second electrode at the second distal end, the first electrode and the second electrode are configured to deliver bipolar radiofrequency energy to the tissue configured to at least one of resect and coagulate tissue. The device is transitionable between a first configuration, in which the first arm and the second arm are configured to grasp tissue between the first end and the second end, and a second configuration, in which at least one of the first arm and the second arm defines scissors configured to mechanically cut tissue. At least one of the first arm and the second arm defines a recess configured to receive a corresponding protrusion on the opposite one of the at least one of the first arm and the second arm. An electrical connector coupled to the proximal ends of both of the first arm and the second arm is included, the electrical connector is configured to electrically couple with a radio frequency generator. At least one moveable finger grip disposed on at least one the first arm and the second arm, wherein movement of the at least one moveable finger grip causes the transition of the device from the first configuration to the second configuration, and when the movable finger grip is in a first position the distal portion of the first arm is locked in a position away from the second arm, and wherein when the moveable finger grip is in a second position the distal portion of the first arm is in contact with the second arm. The first arm includes a proximal portion, a distal portion and a hinge there between, and wherein the distal portion is pivotable with respect to the proximal portion. At least one of the first arm and the second arm defines a fluid pathway therein and a port configured to release a fluid from the fluid pathway.

### BRIEF DESCRIPTION OF THE DRAWINGS

A more complete understanding of the present application, and the attendant advantages and features thereof, will be more readily understood by reference to the following detailed description when considered in conjunction with the accompanying drawings wherein:

FIG. 1 is a front perspective view of an exemplary combination electrosurgical forceps and scissors medical device constructed in accordance with the principles of the present application;

FIG. 2 is a side view of the medical device shown in FIG. 1;

FIG. 3 is a top view of the medical device shown in FIG. 1;

FIG. 4 is a top view of the medical device shown in FIG. 1 with the arms of the forceps in a closed position;

FIG. 5 is a top view of the medical device shown in FIG. 1 with a scissors element disposed proximate the distal end of the arms of the forceps;

FIG. 6 is a top view of the medical device shown in FIG. 5, with the arms of the forceps in a closed position;

FIG. 7 is a top perspective view of an exemplary combination electrosurgical forceps and scissors medical device in a tissue grasping configuration constructed in accordance with the principles of the present application; and

FIG. 8 is a top perspective view of the combination electrosurgical forceps and scissors medical device shown in FIG. 7 in the mechanical cutting configuration.

### DETAILED DESCRIPTION OF THE INVENTION

As used herein, relational terms, such as “first” and “second,” “over” and “under,” “front” and “rear,” and the like, may be used solely to distinguish one entity or element from another entity or element without necessarily requiring or implying any physical or logical relationship or order between such entities or elements.

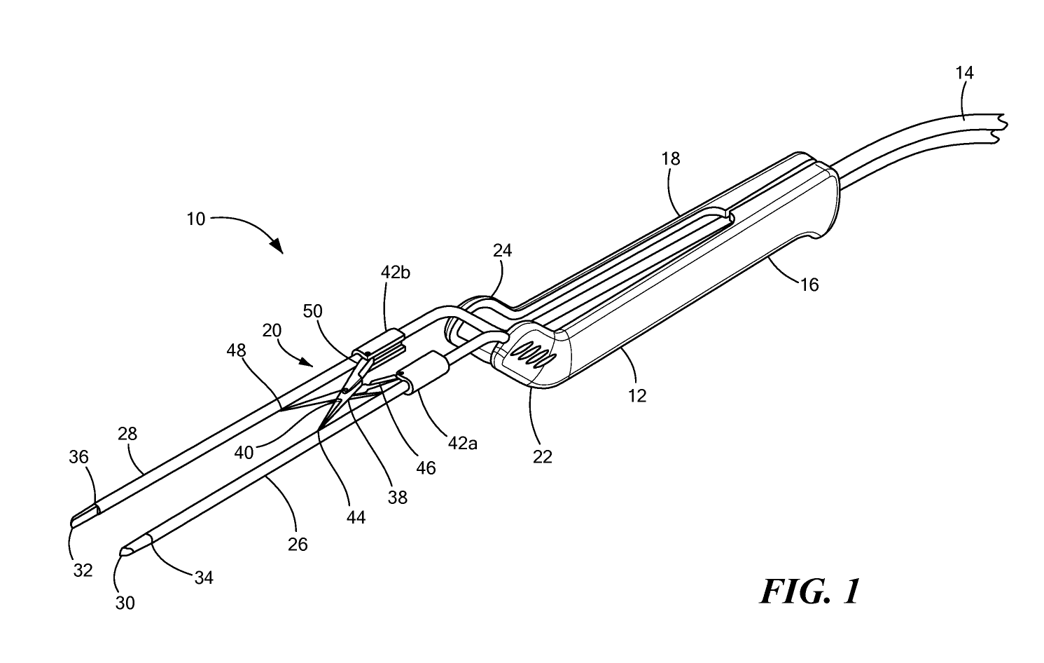

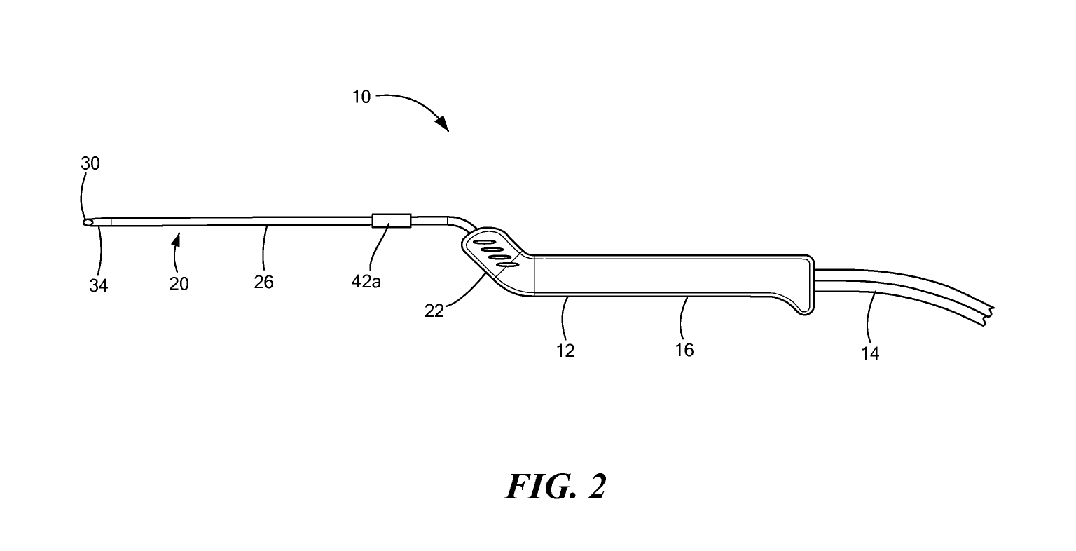

Referring now to the drawings in which like reference designators refer to like elements, there is shown in FIGS. 1-6 an exemplary electrosurgical forceps with an integrated scissors device constructed in accordance with the principles of the present application and designated generally as “10.” The forceps 10 include a handle 12 electrically and fluidly coupled to an electrosurgical unit, for example a radiofrequency generator with integrated fluid supply (not shown) through one or more conduits 14 or connectors. In particular, the electrosurgical unit is configured to controllably deliver a supply of fluid, for example, saline, and to provide electrical power, to the handle 12 of the forceps 10. Each of the conduits 14 may be spilt into two extending through the interior of the handle 12 toward the distal end of handle 12. For example, the handle 12 may include a first handle portion 16 pivotally coupled to a second handle portion 18 at the proximal end of the handle 12. The handle 12 may be biased in an open configuration in which the first handle portion 16 and the second handle portion 18 are spaced a distance away from each other at the distal end of the handle 12. Each handle portion 16 and 18 may define an interior sized to receive the split portion of the conduits 14 such that each handle portion 16 and 18 is configured to deliver electrical power and fluid flow to the effector portion 20 of the device 10. Each handle portion 16 and 18 may define gripping ends 22 and 24 at the respective distal ends of the each handle portion 16 and 18. The gripping ends 22 and 24 may be disposed at an angle with respect to the remainder of each handle portion 16 such that the user may squeeze the gripping ends 22 and 24 together with a combination of a user's index finger and thumb. For example, the gripping ends 22 and 24 may be disposed at an oblique angle, for example, 110 degrees, with respect to the major longitudinal axis of each handle portion 16 and 18. In other configurations, the gripping ends 22 and 24 are substantially parallel with the major longitudinal axis of each handle portion 16 and 18.

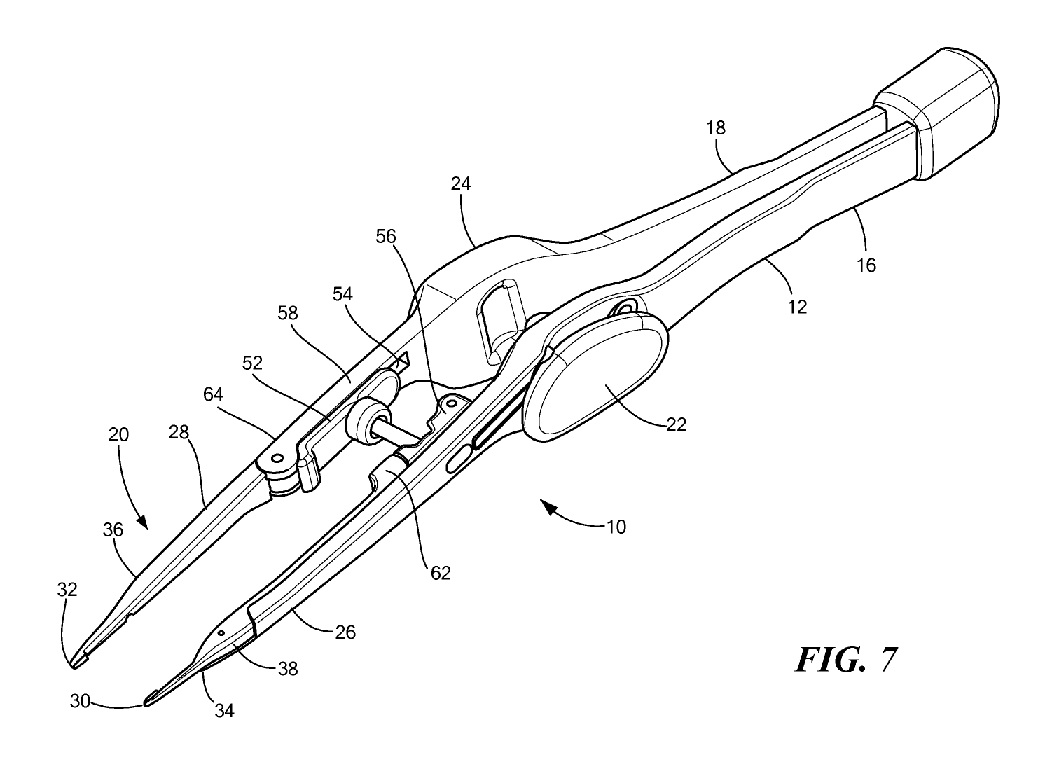

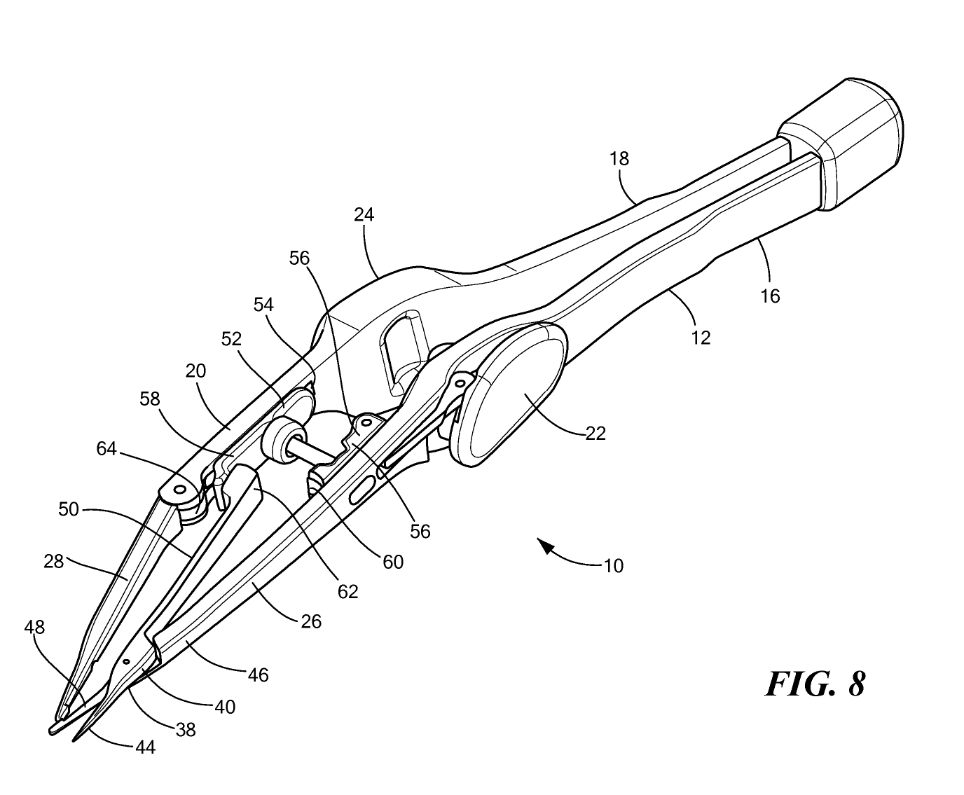

Continuing to refer to FIG. 1, extending from the respective distal ends of the gripping ends 22 and 24 are a first elongate arm 26 and a second elongate arm 28. Each of the arms 26 and 28 may be substantially rigid or malleable and define a hollow interior for passage of one or more conductors (not shown) from the one or more conduits. In particular, each conductor may span the length of each arm 26 and 28 as to provide electrical power to the distal end of the each arm 26 and 28. In an exemplary configuration, the arms 26 and 28 are substantially the same length and in other configurations one arm 26 or 28 may be longer than the other. Each arm 26 and 28 may include one or more fluid lumens (not shown) for the passage of fluid from the conduits 14 to the distal end of each arm 26 and 28. In an exemplary configuration, each arm 26 and 28 is tubular in shape and are substantially parallel with the major longitudinal axis of its respective handle portion 16 and 18. In other configurations, for example as shown in FIGS. 7-8, the arms 26 and 28 are substantially rectangular in shape or flat and may be angled toward each other and may be beveled. Each arm 26 and 28 may include electrical insulation around its respective outer diameter such that they are not electrically conductive along their respective lengths until the distal end of each respective arm 26 and 28. For example, extending from the distal end of each arm 22 and 24 respectively is a first electrically conductive tip 30 and a second electrically conductive tip 32. The tips 30 and 32 may be cylindrical, flat, or rectangular and may define a tapered edge or beveled edge. In one configuration, the tips 30 and 32 define the same outer diameter to that of their respective arms 26 and 28 and in other configurations may define smaller or larger diameters compared to the diameter of their respective arms 26 and 28. In an exemplary configuration the tips 30 and 32 each are flat and define tapered edges as to firm grasp tissue to be treated. The tips 30 and 32 may be composed of electrically conductive material, for example, polished stainless steel, platinum or any of the platinum group metals, and may be electrically charged with a voltage as to delivery electrical energy to the tissue grasped between the tips 30 and 32. For example, the electrosurgical unit may be configured to deliver radiofrequency energy to the tips 30 and 32 sufficient to coagulate and/or cut the tissue grasped between the tips 30 and 32 depending on the amplitude and duty cycle of the radiofrequency energy delivered to the tissue. In an exemplary configuration the tips 30 and 32 are oppositely charged such that bipolar radiofrequency energy may be delivered to the tissue.

The respective distal ends of the arms 26 and 28 may further define a first port 34 and a second port 36 respectively, for the delivery of fluid to the target tissue region. In particular, the distal ends of the arms 26 and 28, or the distal ends of the tips 30 and 32, may define the ports 34 and 36 such that a conductive fluid, such as saline may be delivered to the target tissue. In one configuration, only one port 34 or 36 is included such that fluid exits from only one port. When fluid is delivered to the target tissue region, the tips 30 and 32 may be configured to coagulate the tissue and cause hemostasis of the target tissue at a lower temperature.

Continuing to refer to FIG. 1, slideably coupled between the first arm 26 and the second arm 28 may be a scissors assembly 38. The scissors assembly 38 may include scissors 40 and at least one carrier element 42 slideably coupled to one of the arms 26 and 28. In particular, the scissors 40 may include a first blade 44 having a first leg 46 and a second blade 48 having a second leg 50 pivotally coupled together. The first leg 46 or the second leg 50 may be coupled to the carrier element 42 such that the scissors assembly 38 may be slid from the proximal ends of the arms 26 and 28 to the distal ends of the arms 26 and 28 proximal to the tips 30 and 32. In exemplary configuration, the first leg 46 is coupled to a first carrier element 42a and the second leg 50 is coupled to a second carrier element 42b. Each carrier element 42a and 42b may be at least partially circumferentially disposed about its respective arm 26 and 28 such that each carrier element 42a and 42b may lockingly slide about its respective arm 26 and 28 to any desired longitudinal position. For example, the carrier elements 42a and 42b may slide about each respective arm 26 and 28 by, for example, pinching a tab or a portion of the carrier elements 42a and 42b to loosen each carrier element 42a and 42b from its respective arm 26 and 28 until the pinching force is removed the carrier elements 42a and 42b lock to its respective arm 26 and 28. Thus, in some configurations, a user uses both hands to release and slide each carrier element 42a and 42b in order to move the scissors assembly 38 from one longitudinal position to another. Such a feature may prevent the scissors assembly 38 from inadvertently moving during a procedure. In an exemplary configuration, the scissors 40 is entirely disposed between the arms 26 and 28 such that when then scissor 40 is in an open configuration, as shown in FIG. 1, the blades 44 and 48 are entirely disposed between the arms 26 and 28.

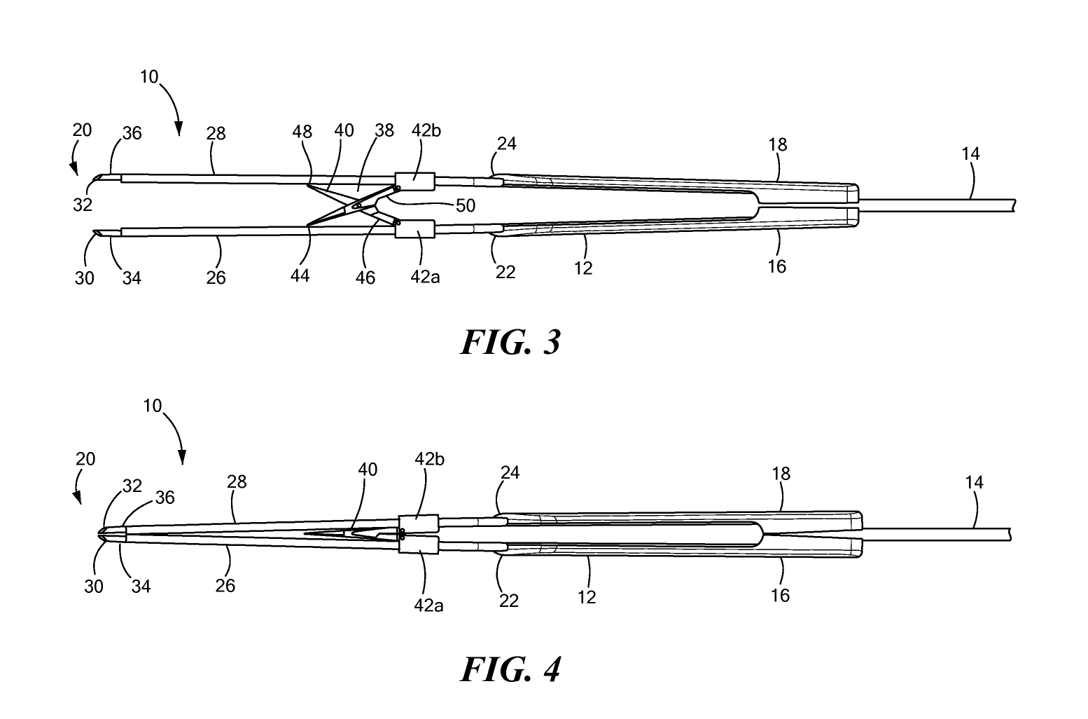

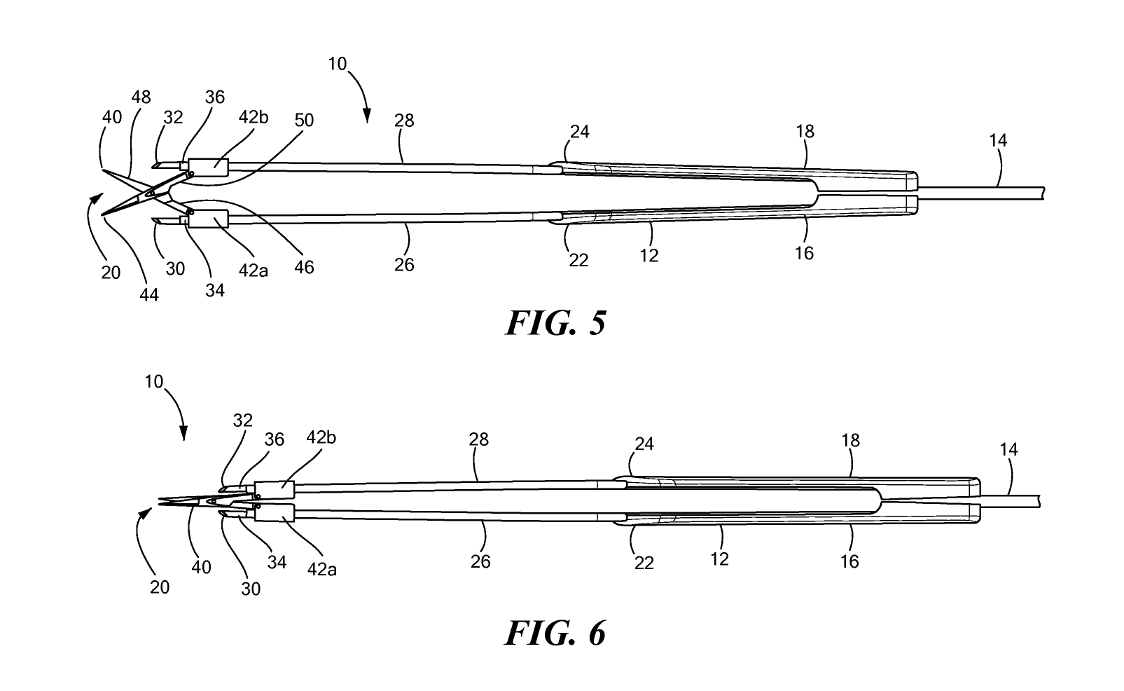

In an exemplary use of the forceps device 10, the user may position the scissors assembly 38 proximal the proximate end of the arms 26 and 28 for using the forceps device 10 to grasp tissue to be treated by bipolar cutting, coagulation, or coagulation with saline. For example, FIGS. 3 and 4 illustrate the device 10 being biased on an open configuration (FIG. 3) and being in a closed configuration (FIG. 4) for using the device to grasp and treat tissue with the tips 30 and 32 of the device as discussed above. During the same or different procedure, the user may slide the scissors assembly 38 in the manner discussed above and lock the carrier elements 42a and 42b at a position proximal to the tips 30 and 32. In such a position, the first blade 44 and the second blade 48 may extend distal to the distal end of the tips 30 and 32, as shown in FIGS. 5 and FIG. 6. The user may use the scissors 40 without interference from the tips 30 and 32 with the pinching of the gripping ends 22 and 24 of the handle 12, which biases the scissors 40 in an open position for easy access to the tissue to be treated. Additionally, the scissors assembly 38 may further operate to seal and/or manipulate tissue. In particular, the scissors assembly 38 being conductive may be in communication with the RF source and may be configuration to conduct bipolar energy between the first blade 44 and the second blade 48.

Referring now to FIGS. 7 and 8 in which an alternative configuration for the forceps device 10 is shown. In this configuration, the scissors 40 (shown expanded in FIG. 8) are formed by the first arm 26, rather than as a separate assembly in the previous embodiment. The gripping ends 22 and 24 may be substantially parallel with the axis defined by the first handle portion 16 and the second handle portion 18. Each gripping end 22 and 24 may include a high friction surface such as rubber or polyurethane such that the user can grip the forceps 10 with a combination of the thumb and index fingers. In this configuration, the user may longitudinally slide, or radially rotate, either or both of the gripping ends 22 and 24 to cause the transition of the forceps 10 to a mechanical cutting device with the scissors assembly 38 without movement of the user's hand position. In particular, the gripping ends 22 and 24 may be movably coupled to a scissors leg engagement element 52. The engagement element 52 may be at least partially slideably received within one or more recesses 54 defined by the first and/or second arms 26 and 28, which allow the engagement element 52 to slide back and forth longitudinally. The engagement element 52 may further include a first end 56 and a second end 58 on opposite arms of the forceps 10. The first end 56 may include a tab 60 extending distally, the tab 60 being configured to releasably engage a notch 62 on the second leg 50 to lock the second leg 50 in a position substantially planar to the first end 56. When the second leg 50 is locked to the first end 56, the first arm 26 and second arm 28 may be moved toward one any other such that the distal ends of the arms 26 and 28 may operate as forceps in the manner discussed above with respect to FIGS. 1-6. In particular, the engagement element 52 may include a telescoping rod 64 disposed between the first end 56 and the second end 58 to facilitate smooth movement of the arms 26 and 28 with respect to each other and/or to provide resistance such that the arms 26 and 28 re-open when a pinching force on the gripping ends 22 and 24 is removed. Moreover, the telescoping rod 64 may assist with the alignment of the distal tips 30 and 32 when the forceps 10 are squeezed. To transition the forceps 10 into a mechanical cutting device, the user actuates one or both of the gripping ends 22 and 24 as discussed above. For example, sliding both first gripping ends 22 and 24 proximally causes proximal movement of the first end 56 and the second end 58. As the first end 56 moves proximally, the second leg 50 is released from the first end 56 and moves toward the second end 58. Simultaneously, as the second end 58 moves proximally, a distal portion of the arm 28 may be released and collapse onto the second blade 48 of the scissors 40, as shown in FIG. 8.

In particular, when the forceps 10 are locked to enable the forceps 10 to grasp tissue, the second end 58 may prevent a hinge 64 from rotating, which in turn, causes the distal portion of the arm 28 to be locked in a substantially parallel position to the first arm 26. When the second end 58 is slid proximally, the hinge 64 is allowed to rotate, which causes movement of the arm 28 toward the scissors assembly 38 to a position proximal to the distal end of the second blade 48. In this configuration, the blades 44 and 48 of the scissors assembly 38 are distal to the arm 28 such that the user can cut tissue without interference of the arm 28. To actuate the scissors 40, pinching of the gripping ends 22 and 24 together, applies a force onto the second leg 50, which is biased against the second end 58, which in turn opens and closes the scissors 40. To switch back to the forceps configuration, the user slides the gripping ends 22 and 24 distally while the first arm 26 and the second arm 28 are pinch together, which locks the tab 60 to the notch 62, to lock the second leg 50 to the arm 26, and which also forces the distal portion of the arm 28 to rotate to a locked position as shown in FIG. 7. Although not shown in FIGS. 7 and 8, it is contemplated that the configuration in FIGS. 7 and 8 further includes a fluid delivery conduit within one or more of the arms 26 and 28 as discussed above, and may include one or more electrodes at the distal ends of the arms 28 and 28, and related ports, such that monopolar or bipolar cutting, coagulation, or coagulation with saline, known as transcollation, may be achieved.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An electrosurgical forceps device, comprising:
a first arm having a first distal end;
a second arm having a second distal end, the first arm and the second arm being pivotally coupled together;
the first arm including a first electrode at the first distal end;
the second arm including a second electrode at the second distal end, the first electrode and the second electrode being configured to deliver bipolar radiofrequency energy to the tissue;
at least one moveable finger grip disposed on at least one the first arm and the second arm; and
the device being transitionable between a first configuration, in which the first arm and the second arm are configured to grasp tissue between the first end and the second end, and a second configuration, in which at least one of the first arm and the second arm defines scissors configured to mechanically cut tissue.

2. The device of claim 1, wherein the first electrode and the second electrode are configured to at least one of resect and coagulate tissue.

3. The device of claim 1, wherein movement of the at least one moveable finger grip causes the transition of the device from the first configuration to the second configuration.

4. The device of claim 3, wherein the first arm includes a proximal portion, a distal portion and a hinge there between, and wherein the distal portion is pivotable with respect to the proximal portion.

5. The device of claim 4, wherein when the movable finger grip is in a first position the distal portion of the first arm is locked in a position away from the second arm, and wherein when the moveable finger grip is in a second position the distal portion of the first arm is in contact with the second arm.

6. The device of claim 1, wherein at least one of the first arm and the second arm defines a fluid pathway therein and a port configured to release a fluid from the fluid pathway.

7. The device of claim 1, wherein the entirety of the scissors is defined by the at least one of the first arm and the second arm.

8. The device of claim 8, wherein the scissors is disposed between the first arm and the second arm.

9. The device of claim 1, further including an engagement element disposed between the first arm and the second arm proximate the at least one moveable finger grip, the engagement element being configured to bias the first arm away from the second arm.

10. The device of claim 9, wherein the engagement element includes a biasing member between the first arm and the second arm.

11. The device of claim 1, wherein at least one of the first arm and the second arm defines a recess configured to receive a corresponding protrusion on the opposite one of the at least one of the first arm and the second arm.

12. The device of claim 1, further including an electrical connector coupled to the proximal ends of both of the first arm and the second arm, the electrical connector being configured to electrically couple with a radiofrequency generator.

13. An electrosurgical forceps device, comprising:
a first arm having a first distal end;
a second arm having a second distal end, the first arm and the second arm being pivotally coupled together;
the first arm including a first electrode at the first distal end;
the second arm including a second electrode at the second distal end, the first electrode and the second electrode being configured to deliver bipolar radiofrequency energy to the tissue; and
a scissors assembly movably coupled to at least a portion of the first arm and at least a portion of the second arm.

14. The device of claim 13, wherein the scissors assembly is disposed between the first arm and the second arm.

14. The device of claim 14, wherein the scissors assembly includes a first carrier element slideably coupled to the first arm and a second carrier element slideably coupled to the second arm, and wherein the scissors assembly includes scissors coupled to the first carrier element and the second carrier element.

16. The device of claim 15, wherein the device is transitionable between a first configuration, in which the first arm and the second arm are configured to grasp tissue between the first end and the second end, and a second configuration, in which the scissors extends beyond the first end and the second end and is configured to mechanically cut tissue.

17. The device of claim 16, further defining a fluid flow pathway disposed within it least one of the first arm and the second arm, and wherein at least one of the first arm and the second arm define a port, the port being in fluid communication with the fluid flow pathway.

18. The device of claim 17, wherein the scissors are biased in an open configuration in which the first arm is a predetermined distance away from the second arm, and wherein movement of the first arm toward the second arm transitions the scissors from an open configuration to a closed configuration.

19. An electrosurgical forceps device, comprising:
a first arm having a first distal end;
a second arm having a second distal end, the first arm and the second arm being pivotally coupled together;
the first arm including a first electrode at the first distal end;
the second arm including a second electrode at the second distal end, the first electrode and the second electrode being configured to deliver bipolar radiofrequency energy to the tissue configured to at least one of resect and coagulate tissue;
the device being transitionable between a first configuration, in which the first arm and the second arm are configured to grasp tissue between the first end and the second end, and a second configuration, in which at least one of the first arm and the second arm defines scissors configured to mechanically cut tissue;
at least one of the first arm and the second arm defines a recess configured to receive a corresponding protrusion on the opposite one of the at least one of the first arm and the second arm;
an electrical connector coupled to the proximal ends of both of the first arm and the second arm, the electrical connector being configured to electrically couple with a radio frequency generator;
at least one moveable finger grip disposed on at least one the first arm and the second arm, wherein movement of the at least one moveable finger grip causes the transition of the device from the first configuration to the second configuration, and when the movable finger grip is in a first position the distal portion of the first arm is locked in a position away from the second arm, and wherein when the moveable finger grip is in a second position the distal portion of the first arm is in contact with the second arm. the first arm includes a proximal portion, a distal portion and a hinge there between, and wherein the distal portion is pivotable with respect to the proximal portion; and
at least one of the first arm and the second arm defines a fluid pathway therein and a port configured to release a fluid from the fluid pathway.
